(19) United States
(12) Patent Application Publication
(10) Pub. No.: US 2026/0034329 A1
(43) Pub. Date: Feb. 5, 2026

(54) PORTABLE GAS SOURCE CONTROL SYSTEM FOR VENTILATION THERAPY

(71) Applicant: HILL-ROM SERVICES PTE. LTD., SINGAPORE (SG)

(72) Inventors: Tom Westfall, Irvine, CA (US); Gary LaTorraca, Huntington Beach, CA (US)

(21) Appl. No.: 19/210,563

(22) Filed: May 16, 2025

Related U.S. Application Data
(60) Provisional application No. 63/679,483, filed on Aug. 5, 2024.

(51) Int. Cl. A61M 16/10; A61M 16/00; A61M 16/20; G16H 40/63

(52) U.S. Cl. CPC A61M 16/1005; A61M 16/024; A61M 16/202; G16H 40/63; A61M 2202/0007; A61M 2202/0208; A61M 2205/3327; A61M 2205/3331; A61M 2205/502; A61M 2230/42

(57) ABSTRACT

A method of controlling a portable gas source connected to a ventilator includes receiving, via a user interface, a mix setting corresponding to a user-selected percentage of oxygen in a mixture of oxygen and ambient air to be delivered by the ventilator, determining, based on a measured airflow demand on the ventilator, a maximum percentage of oxygen in the mixture at which the mixture still meets the measured airflow demand, and overriding the mix setting with the maximum mix value in response to the maximum mix value being less than the user-selected percentage.

### CROSS-REFERENCE TO RELATED APPLICATIONS

The present application claims priority to U.S. Provisional Application Ser. No. 63/679,483 filed Aug. 5, 2024, the disclosure of which is incorporated herein by reference.

### STATEMENT RE: FEDERALLY SPONSORED RESEARCH/DEVELOPMENT

Not Applicable

### BACKGROUND

The present disclosure relates generally to ventilation therapy and, more particularly, to a control system for a portable gas source.

A wide range of clinical conditions may require some form of ventilation therapy, whereby the patient's work of breathing is assisted by the flow of pressurized gas from a ventilator to the patient's airway. These conditions may include hypoxemia, various forms of respiratory insufficiency, and airway disorders. There are also non-respiratory and non-airway diseases that require ventilation therapy, such as congestive heart failure and neuromuscular diseases.

To improve the quality of life of many patients who require long-term ventilation therapy, ventilation systems have been developed which are miniaturized and portable. Some of these systems, for example, the Life2000® system by Breathe Technologies, Inc., are so lightweight and compact that in their extended range or stand-alone configurations, they are wearable by the patient. These systems make use of a source of pressurized ventilation gas to operate. In the stationary or extended-range configuration, the source of pressurized gas may be a stationary compressor unit, which may be kept in a patient's home. In the stand-alone configuration, which may be generally used when the patient is outside the home, the portable, wearable ventilator generally receives its ventilation gas from a pressurized gas cylinder or a portable compressor.

Many of the above clinical conditions and other clinical conditions may also require or benefit from supplemental oxygen therapy, whereby the gas introduced to the patient's airway is augmented by the presence of additional oxygen such that the patient inspires gas having oxygen levels above atmospheric concentration (20.9% at 0% humidity). Supplemental oxygen therapy involves the patient receiving supplemental oxygen gas from an oxygen gas source, which is typically a compressed or cryogenic oxygen cylinder, or an oxygen gas generator. For many years, patients who wished to be mobile relied on oxygen cylinders. However, in recent years, miniaturization and improvements in battery technology has resulted in the development of portable oxygen concentrators.

Portable oxygen concentrators typically operate by pressure swing adsorption (PSA), in which ambient air is pressurized by a compressor and passed through an adsorbent sieve bed. The sieve bed is typically formed of a zeolite which preferentially adsorbs nitrogen when at high pressure while oxygen passes through. Once the sieve bed reaches its capacity to adsorb nitrogen, the pressure can be reduced. This reduction in pressure causes the adsorbed nitrogen to be desorbed so it can be purged, leaving a regenerated sieve bed that is again ready to adsorb nitrogen. With repeated cycles of this operation, an enriched oxygen gas may be generated. Typically, portable oxygen concentrators have at least two sieve beds so that one may operate while the other is being purged of the nitrogen and vented. Typical portable oxygen concentrators today output an enriched oxygen gas with a purity of around 87-96% oxygen. Among existing oxygen concentrators today which may be considered portable (especially by an individual suffering from a respiratory condition), there are generally two types available. The first type, which is larger and heavier, is usually capable of continuous flow delivery. Models of this type typically weigh between 5-10 kg, have maximum flow rates of around 5-6 liters per minute or less, and are generally configured with wheels and a handle, often mimicking the appearance of a suitcase. The second type are lighter units more suitable for being carried or worn in a satchel, handbag, or a backpack. Models of this type typically weigh less than 2.5 kg and are usually limited to pulsed delivery modes with maximum flow rates of around 2 liters per minute or less.

Portable oxygen concentrators have a substantial cost and convenience advantage over pressurized oxygen cylinders, due to the pressurized oxygen cylinders requiring ongoing refilling or replacement. Additionally, portable oxygen concentrators are considered to be significantly safer than pressurized oxygen cylinders. This safety consideration can have a substantial impact on a patient's quality of life, because many portable oxygen concentrators have been approved by the FAA for use by travelers on commercial airlines, whereas oxygen cylinders are universally banned on commercial flights. Consequently, patients with pressurized oxygen cylinders must make expensive and time-consuming preparations with an airline ahead of time or forego airline travel entirely.

For patients with conditions where assistance with the work of breathing is not required, supplemental oxygen therapy alone, without ventilation therapy, may be sufficient. However, for many patients, combined ventilation therapy and supplemental oxygen therapy may be a more optimal treatment. In healthy patients, sufficient ventilation to perform the work of breathing may typically require minute ventilation rates of between 5 and 8 L/min while stationary, which may double during light exercise, and which may exceed 40 L/min during heavy exercise. Patients suffering from respiratory conditions may require substantially higher rates, and substantially higher instantaneous rates. This is especially true when these patients are outside the home and require portability, as at these times such patients are often also involved in light exercise.

It may thus be seen that patients who would prefer to receive this combined mode of treatment are substantially limited, since in many cases existing portable oxygen concentrators do not output gas at pressures and/or volumes high enough to be used with a wearable, portable ventilator without the presence of an additional source of compressed gas. While existing systems and methods that seek to provide a combined supplemental oxygen/ventilation system have been developed in the prior art, these existing systems suffer from various deficiencies which Applicant has addressed in the system described in its U.S. Pat. No. 11,607,519 (“the '519 patent”), entitled O2 “CONCENTRATOR WITH SIEVE BED BYPASS AND CONTROL METHOD THEREOF,” the entire disclosure of which is incorporated by reference herein.

When developing control algorithms for a portable gas source (PGS) connected to a ventilator such as the PGS described in the '519 patent, the safety of the patient is of the utmost importance. In the case of source gas insufficiency, for example, measures must be taken to ensure that adequate airflow to the patient is maintained.

### BRIEF SUMMARY

The present disclosure contemplates various systems and methods for overcoming the above drawbacks accompanying the related art. One aspect of the embodiments of the present disclosure is a computer program product comprising one or more non-transitory program storage media on which are stored instructions executable by a processor or programmable circuit to perform operations for controlling a portable gas source connected to a ventilator. The operations may comprise receiving, via a user interface, a mix setting corresponding to a user-selected percentage of oxygen in a mixture of oxygen and ambient air to be delivered by the ventilator, determining, based on a measured airflow demand on the ventilator, a maximum percentage of oxygen in the mixture at which the mixture still meets the measured airflow demand, and overriding the mix setting with the maximum mix value in response to the maximum mix value being less than the user-selected percentage.

Another aspect of the embodiments of the present disclosure is a method of controlling a portable gas source connected to a ventilator. The method may comprise receiving, via a user interface, a mix setting corresponding to a user-selected percentage of oxygen in a mixture of oxygen and ambient air to be delivered by the ventilator, determining, based on a measured airflow demand on the ventilator, a maximum percentage of oxygen in the mixture at which the mixture still meets the measured airflow demand, and overriding the mix setting with the maximum mix value in response to the maximum mix value being less than the user-selected percentage.

Another aspect of the embodiments of the present disclosure is a system for controlling a portable gas source connected to a ventilator. The system may comprise a user interface by which a user may input a mix setting corresponding to a user-selected percentage of oxygen in a mixture of oxygen and ambient air to be delivered by the ventilator and one or more processors operable to determine, based on a measured airflow demand on the ventilator, a maximum percentage of oxygen in the mixture at which the mixture still meets the measured airflow demand and to override the mix setting with the maximum mix value in response to the maximum mix value being less than the user-selected percentage. The user interface may be provided on a housing of the ventilator or portable gas source. The one or more processors may be provided in the ventilator and/or portable gas source.

In any of the above aspects, determining the maximum percentage of oxygen in the mixture at which the mixture still meets the measured airflow demand may include calculating a total volume being demanded in one period based on a breath rate and a delivered bolus size and determining the maximum percentage of oxygen based on the calculated total volume. The operations or method may comprise calculating, based on the maximum mix value, a pressure target for a storage tank of the mixture of oxygen and ambient air to be delivered by the ventilator. The operations or method may comprise calculating, based on the maximum mix value and the pressure target, a total volume of gas needed to fill the product tank. The operations or method may comprise calculating, based on the maximum mix value and the total volume of gas needed to fill the product tank, a total volume of air needed to fill the product tank and a total volume of oxygen needed to fill the product tank. The operations or method may comprise controlling a solenoid valve based on the total volume of air needed to fill the product tank and the total volume of oxygen needed to fill the product tank.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a high-level diagram of a ventilation therapy system according to an embodiment of the present disclosure;

FIG. 2 is a schematic diagram of a portable gas source (PGS) of the ventilation therapy system;

FIG. 3 is a schematic diagram of a ventilator of the ventilation therapy system; and

FIG. 4 is a functional block diagram depicting an oxygen blending control system and operation thereof in relation to the ventilation therapy system.

### DETAILED DESCRIPTION

The present disclosure encompasses various embodiments of systems and methods for controlling a portable gas source for use in ventilation therapy. The detailed description set forth below in connection with the appended drawings is intended as a description of several currently contemplated embodiments and is not intended to represent the only form in which the disclosed subject matter may be developed or utilized. The description sets forth the functions and features in connection with the illustrated embodiments. It is to be understood, however, that the same or equivalent functions may be accomplished by different embodiments that are also intended to be encompassed within the scope of the present disclosure. It is further understood that the use of relational terms such as first and second and the like are used solely to distinguish one from another entity without necessarily requiring or implying any actual such relationship or order between such entities.

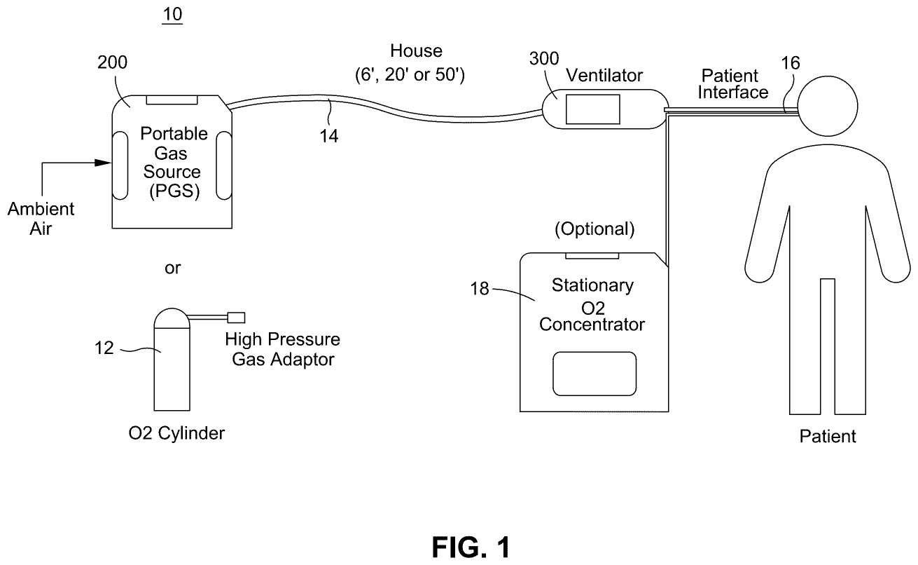

FIG. 1 is a high-level diagram of a ventilation therapy system 10 according to an embodiment of the present disclosure. The ventilation therapy system 10 may provide mobile non-invasive ventilation to critically ill homecare and hospital patients. In an exemplary homecare configuration, the ventilation therapy system 10 may include a gas source 12, 200, a hose 14, a ventilator 300, a patient interface 16, and an optional stationary O₂ concentrator 18. The gas source can be either a portable gas source (PGS) 200 as described in more detail below, a stationary gas source (SGS), or a high pressure O₂ cylinder 12, for example. The PGS 200 (or SGS) may provide pressurized mixed gas to the ventilator 300 via the hose 14. The pressurized mixed gas may be at 15 to 22 psi, for example. The gas mixture may be from 21% to 95% oxygen based on a user selected setting on the PGS 200 (or SGS). An O₂ cylinder 12 with a high-pressure gas adaptor (HPGA) can also be used to provide 100% O₂ via the hose 14 to the ventilator 300 (e.g., at 25 psi).

The ventilator 300 may, for example, be a ventilator as described in U.S. Pat. No. 10,369,320 (“the '320 patent”), entitled “MODULAR VENTILATION SYSTEM,” the entire disclosure of which is incorporated by reference herein. The ventilator 300 may differ from the ventilator described in the '320 patent in that it may require lower inlet pressures. The ventilator 300 may take in high pressure mixed gas from 8 to 25 psi and deliver positive pressure volume ventilation to a patient via a venturi-based patient interface 16 such as an interface described in U.S. Patent Application Pub. No. 2022/0339378 (“the '378 publication”), entitled “ACCURATE PRESSURE MEASUREMENT WITH NON-INVASIVE VENTILATION NASAL PILLOWS,” the entire disclosure of which is incorporated by reference herein. The patient interface 16 may amplify the ventilator gas by approximately 3:1, thus delivering peak flows from 18 to 120 lpm of gas to the patient.

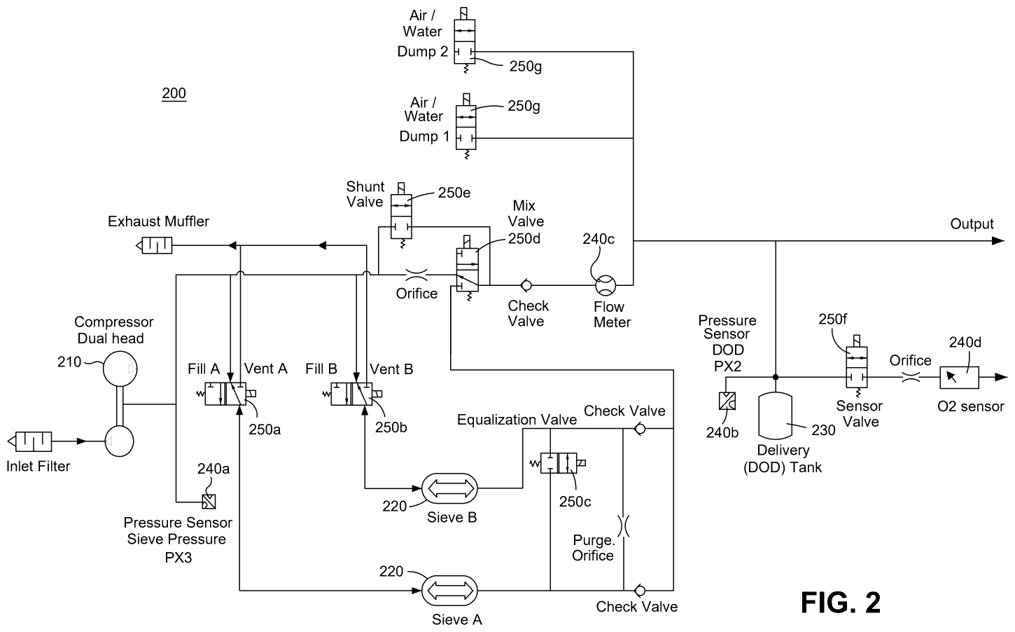

FIG. 2 is a schematic diagram of an exemplary portable gas source (PGS) 200 (or stationary gas source SGS) of the ventilation therapy system 10. The PGS 200 may, for example, be an oxygen concentrator as described in the '519 patent and may be arranged to deliver a high oxygen content gas produced by the PGS 200 to a patient via the patient ventilation interface 16. Depending on various factors including, for example, the prescription of the patient, the patient's activity level, user-adjustable settings, and the state of the patient's breathing in a given moment, the ventilator 300 may instruct the PGS 200 to produce a specific flow (e.g., volume flow) of gas having a specific oxygen concentration. The ventilator 300 may then provide such high oxygen content gas to the patient via the patient ventilation interface 16 such that, taking into account any entrainment of additional ambient air in the patient ventilation interface 16, the patient is provided with a desired degree of assistance to the patient's work of breathing and a target FiO₂. In order to produce the high oxygen content gas from ambient air, a compressor 210 of the PGS 200 may pump ambient air through one or more adsorbent sieve beds 220 that remove nitrogen from the pressurized air. The resulting gas having high oxygen concentration (e.g. >90%) may then flow into a product tank 230 for delivery to the ventilator 300. In more detail, a controller of the PGS 200 may control one or more valves in order to cyclically bring pressurized ambient air into the sieve bed(s) 220 and exhaust the nitrogen waste product extracted by the sieve bed(s) 220. Two sieve beds 220 may be provided (e.g., Sieve A and Sieve B as shown in FIG. 2) having opposed operation cycles, Sieve A filling the product tank 230 with high oxygen content gas at the same time that Sieve B is exhausting nitrogen to ambient and vice versa.

More specifically, the PGS 200 may take in ambient air and compress the air to 15 to 22 psi via a compressor 210 such as a dual piston compressor pump. The output of the compressor 210 may be measured by a sieve pressure sensor 240a, PSieve, and may go to an oxygen concentrator subsystem which consists of two three-way solenoids 250a, 250b attached to sieve beds A and B, and an equalization solenoid valve 250c. The oxygen concentrator subsystem may use the solenoid valves 250a, 250b, 250c to alternatively fill and vent the sieve beds 220 using a Pressure Swing Adsorption (PSA) process. Zeolite in the sieve beds adsorbs the nitrogen from the compressed air, and the remaining output gas may be, for example, 95% oxygen at 15 to 22 psi. The oxygen goes to the delivery tank or product tank 230 via a mix solenoid valve 250d. The system may, for example, create a maximum of 1.1 L of 95% oxygen at 15 to 22 psi. It may take approximately 13.5 lpm of compressed air to produce 1.1 L of 95% oxygen. The remaining nitrogen in the sieve beds may be vented to atmosphere through an exhaust muffler as shown.

The compressor 210 may be capable of producing up to 17 lpm of air at 25 psi. The compressor output goes to both the mix solenoid valve 250d as well as the oxygen concentrator subsystem via the valves 250a, 250b. The mix solenoid 250d may alternately fill the delivery tank 230 from both the air and oxygen legs of the PGS 200 resulting in the delivery tank 230 containing a patient selected O₂ concentration from 21% to 95% oxygen. When the PGS 200 is set to 21% oxygen, all the air from the compressor 210 is sent to the storage tank 230. In this case, the mix solenoid may be completely bypassed via a shunt solenoid valve 250e that has a much larger orifice in order to reduce the pressure drop from the higher flow of gas. As described herein, regardless of the user setting for oxygen concentration, the PGS 200 may advantageously maintain product tank pressure as measured by the tank pressure sensor 240b, PTank, by adding air to the product tank 230 when ventilator demand is high. This is done so that the ventilator 300 always has enough gas to meet a patient's demand and the patient is never air starved. At high ventilator demand the delivered oxygen gas concentration may fall below the set oxygen level.

The accuracy of the mixed gas may be achieved by using a flow sensor 240c just downstream of the mix solenoid 250d. Since the system knows the state of the mix solenoid 250d, the measurement by the flow sensor 240c can be used to determine both the volume of air and the volume of oxygen that is being delivered to the storage tank 230. In addition, there may be an oxygen sensor 240d and solenoid 250f teed into the storage tank 230 that is used to periodically measure the percent oxygen in the storage tank 230. This information may be used both for fine tuning the blending algorithm and for a low O₂ alarm. There may also be two water/dump solenoid valves 250g that are used to periodically purge any water that has accumulated in the bottom of the delivery tank 230. The delivery tank 230 may be approximately 630 ml and may provide enough gas to deliver the peak flow, e.g., 6 to 40 lpm, required for any breath. This allows the compressor 210 and oxygen concentrator subsystem of the PGS 200 to provide the average flow or minute volume that a patient requires instead of the peak flows required. In addition, the patient interface may further amplify the flow that the patient will receive, such that the PGS 200 may only need to provide one-third of the patient minute volume. For example, if the patient needs 6 L minute volume, 500 ml at 12 bpm, the PGS 200 would only need to provide 2 L of flow over one minute due to the patient interface amplifying the flow by 3:1 and the delivery tank 230 providing the required peak flow.

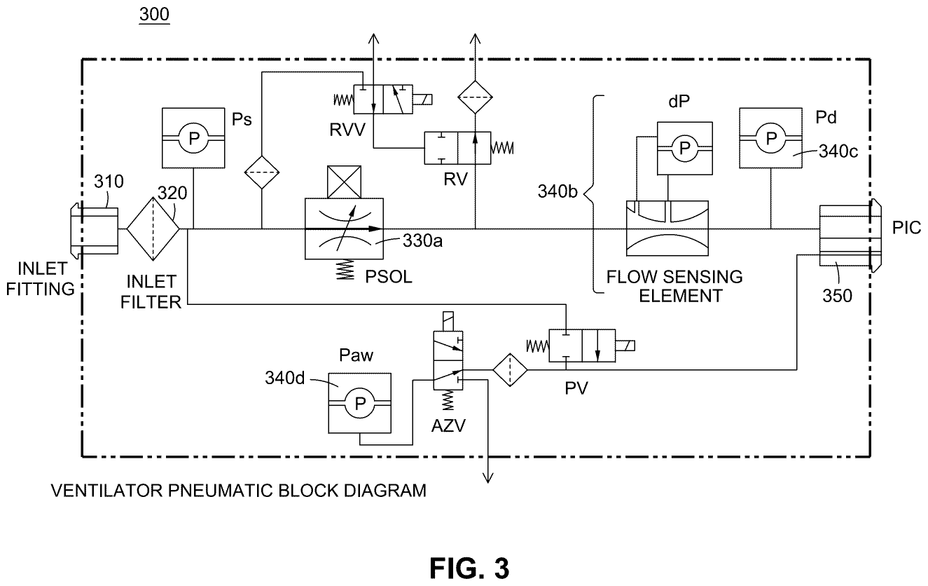

FIG. 3 is a schematic diagram of a ventilator 300 of the ventilation therapy system 10. The ventilator 300 may accept pressurized gas, e.g., 8 to 25 psi, from an external gas source such as the PGS 200 (or SGS) of the ventilation therapy system 10. The gas may be delivered to the ventilator 300 via the gas hose 14 shown in FIG. 1, for example, which may be connected to the inlet fitting 310. The gas may be filtered by a 40-micron inlet filter 320 to protect both a proportional solenoid valve PSOL 330a and the patient. A pressure sensor Ps 340a may measure the inlet pressure to detect a low or high inlet source pressure. The delivered flow of gas to the patient may be controlled by the PSOL 330a and a downstream flow sensor 340b. The PSOL 330a may be capable of delivering from 0 to 40 lpm of flow, and the flow sensor 340b may measure the delivered flow from the PSOL 330a. An electronic PI filter may use the output from the flow sensor 340b to control the PSOL 330a based on user settings. The flow sensor 340b may have a fixed orifice flow sensing element and a delta pressure sensor, dP, to measure the pressure drop across the flow sensing element. The system may be calibrated during production to create a look up table of dP versus flow. Just downstream from the flow sensor 340b there may be a delivered pressure sensor Pd 340c. This sensor is used to determine circuit disconnects and occlusions. Normal delivered pressure may vary from 0.5 to 16.6 psi based on the flow.

The system may also contain an airway pressure sensor Paw 340d which is used to measure the pressure in the patient's lungs. The patient circuit or interface 16 (see FIG. 1) may be a dual lumen system, with one lumen containing the delivered gas to the patient which comes from the PSOL 330a and a second lumen used to measure the patient airway pressure via the Paw sensor 340d. The patient interface connector PIC 350 may be used to connect the patient interface 16 to the ventilator 300, both providing a connection such that the patient pressure sense lumen is connected to the Paw sensor 340d via the patient interface connector PIC 350. The Paw sensor 340d may also have an autozero solenoid, AZV, to periodically zero the Paw sensor 340d. In addition, there may be a purge solenoid valve, PV, which may be used to periodically purge the pressure sense lumen in the patient circuit.

There may be various kinds of patient interface 16 that may be alternately used depending on the needs of the patient. Exemplary patient interfaces 16 may include nasal pillows interfaces, cannulas, and universal circuit connectors (UCC) as variously described in the '378 publication, the '320 patent, U.S. Patent Application Pub. No. 2022/0249797, entitled “VARIABLE THROAT JET VENTURI,” U.S. Patent Application Pub. No. 2022/0126052, entitled “MULTIFUNCTIONAL VENTILATOR INTERFACES,” U.S. Pat. No. 10,792,449, entitled “PATIENT INTERFACE WITH INTEGRATED JET PUMP,” and U.S. Pat. No. 10,307,552, entitled “JET PUMP ADAPTOR FOR VENTILATION SYSTEM,” the entire disclosure of each of which is incorporated by reference herein. The patient interface 16 may connect to the ventilator 300 via the patient interface connector (PIC) 350, which provides a connection for both the patient gas and a sense line used to measure the patient's airway pressure (Paw) as described above. The patient interface 16 may receive high pressure gas, e.g., 0 to 16.6 psi, from the ventilator 300 via a patient gas lumen of the patient interface 16. This gas may be delivered to the patient via one or more jet nozzles. The high-pressure gas may exit the jet nozzle(s) at high velocity and entrain additional gas via one or more entrainment ports to deliver a total gas to the patient that is approximately 3 to 4 times larger than the flow from the ventilator 300. In addition, the high velocity gas from the jet(s) may create a positive pressure in the patient airway of, for example, up to 55 cmH2O at 30 lpm of V'n (nozzle flow). During exhalation, the high-pressure gas in the patient lumen may be shut off by the ventilator 300, thus allowing the patient to exhale through the entrainment port(s). By way of example, at 30 lpm of drive flow from the ventilator 300, the stagnation pressure may be 55 cmH2O and total patient flow may be greater than or equal to 105 lpm when there is zero back (patient) pressure.

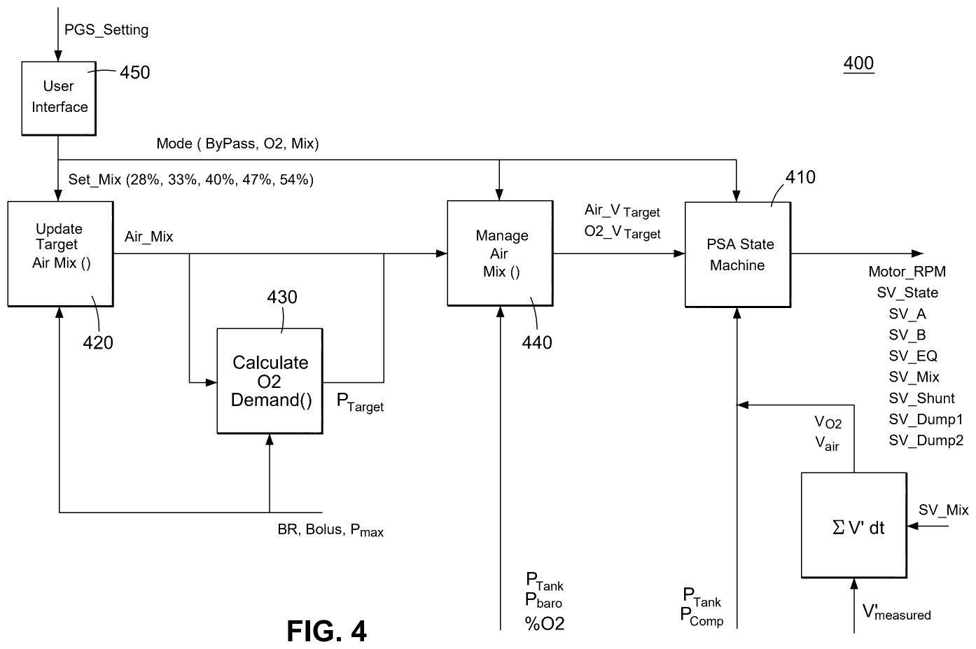

FIG. 4 is a functional block diagram depicting an oxygen blending control system 400 and operation thereof according to an embodiment of the present disclosure. The oxygen blending control system 400 may determine the operational state of the portable gas source 200 in relation to the PSA cycle of the sieve beds 220 based on user input as well as sensor inputs received from the various sensors associated with the ventilation therapy system 10. Advantageously, the control system 400 may control the PGS 200 so as to meet both airflow and oxygenation demands of the system 10 with priority given to airflow over oxygenation in order to ensure the safety of the patient in the event of any source gas insufficiency.

The control of the PSA cycle may be represented as a PSA state machine 410, an example of which is set forth in Table 1, below:

TABLE 1

| State | Exit Criteria |
|---|---|
| Init Fill | One Tick |
| Fill | Time Out or |
| | (P_{TANK} > P_{TARGET}) & (P_{COMP} > P_{TARGET}) |
| EQU_Upper_Int | One Tick |
| EQU_Upper | 0.3 Seconds |
| EQU_Lower_Int | One Tick |
| EQU_Lower | 0.5 Seconds |

The PSA cycle may have three functional states, FILL, EQU_Upper, and EQU_Lower, along with three corresponding initialization states, Init Fill, EQU_Upper_Int, and EQU_Lower_t. The initialization states may be used to set valve states and to start or stop relevant timers as described herein in association with the corresponding functional states. The PSA state machine 410 may cycle through these six states twice (one for each sieve bed 220) to complete one full PSA cycle. The PSA cycle states may define valve positions and compressor motor speed (rpm) for each of a bypass mode, an O₂ mode, and a mix mode of the PGS 200 as set forth in Table 2, below:

TABLE 2

| | | Sieve | Sieve | EQU | Mix | Shunt | O₂ | Dump | Dump | Motor |
|---|---|---|---|---|---|---|---|---|---|---|
| State | Mode | A SV | B SV | SV | SV | SV | SV | 1 SV | 2 SV | rpm |
| Init | Bypass | Fill | Fill | Closed | Air | On | P | Closed | Closed | Constant |
| Fill | O₂ | Fill | Vent | Closed | O₂ | Closed | P | Closed | Closed | Constant |
| | Mix | Fill | Vent | Closed | Mix | Closed | P | Closed | Closed | Constant |
| Fill | Bypass | Fill | Fill | Closed | Air | On | P | Closed | Closed | Constant |
| | O₂ | Fill | Vent | Closed | O₂ | Closed | P | Closed | Closed | Constant |
| | Mix | Fill | Vent | Closed | Mix | Closed | P | Closed | Closed | Constant |
| EQU | Bypass | Fill | Fill | Closed | Air | On | P | Closed | Closed | Constant |
| Upper | O₂ | Fill | Vent | Open | O₂ | Closed | P | Closed | Closed | Constant |
| Int | Mix | Fill | Vent | Open | Last | Closed | P | Closed | Closed | Constant |
| EQU | Bypass | Fill | Fill | Closed | Air | On | P | Closed | Closed | Constant |
| Upper | O₂ | Fill | Vent | Open | O₂ | Closed | P | Closed | Closed | Constant |
| | Mix | Fill | Vent | Open | Last | Closed | P | Closed | Closed | Constant |
| EQU | Bypass | Fill | Fill | Closed | Air | On | P | Open | Open | Constant |
| Lower | O₂ | Fill | Fill | Open | O₂ | Closed | P | Closed | Closed | Constant |
| Int | Mix | Fill | Fill | Open | Last | Closed | P | Closed | Closed | Constant |
| EQU | Bypass | Fill | Fill | Closed | Air | On | P | Closed | Closed | Constant |
| Lower | O₂ | Fill | Fill | Open | O₂ | Closed | P | Closed | Closed | Constant |
| | Mix | Fill | Fill | Open | Last | Closed | P | Closed | Closed | Adjust* |

An exemplary PSA cycle during the O₂ mode or the mix mode may start in a FILL state. Sieve-A solenoid valve 250a is put in the fill position so that sieve-A is filled with air from the compressor 210. Sieve-B solenoid valve 250b is put in the vent state so that all gas in sieve-B is vented to atmosphere through the exhaust muffler. The equalization solenoid valve 250c is closed so that no gas can be exchanged between the sieve beds 220 except for a small amount of oxygen that will go through the purge orifice. As indicated in Table 1, the system will stay in the FILL state until the pressure in both the storage tank 230 and sieve A reaches the target pressure (i.e., P_{Tank}>P_{Target} & P_{Comp}>P_{Target}), or 10 seconds elapses (i.e., “Time Out,” which may be 1.5 seconds in bypass mode), whichever occurs first. The system attempts to fill sieve A in approximately 6 seconds (or other predetermined length of time, which may be 1 second in bypass mode). This is controlled by the speed of the compressor 210 which is adjusted at the end of the PSA cycle. As sieve A is filled with air the zeolite material will adsorb the nitrogen from the air and the remaining gas that exits the sieve bed is 95% oxygen.

Once the FILL state is complete, the system will transition to the EQU_Upper state. Sieve-A and Sieve-B solenoid valves 250a, 250b remain in their respective Fill and Vent states, but the Equalization solenoid valve 250c is opened for 0.3 seconds. This allows the nitrogen from both Sieve A and B to be dumped through the exhaust muffler via sieve bed B. This is the first part of the purging and equalization process. Purging is necessary for the zeolite to release the nitrogen it has absorbed from the previous FILL cycle.

After 0.3 seconds the system enters the EQU_Lower state. Both sieve-A and sieve-B solenoid valves 250a, 250b are set to fill for 0.5 seconds. The gas from the compressor 210 will go to the sieve bed 220 with the lowest pressure. After the 0.5 seconds both sieve beds 220 will have the same pressure, which will be approximately half of the target pressure.

From here the system will go into the second part of the PSA cycle. It is the same six states as cycle 1 (three distinct functional states with three corresponding initialization states) except sieve-A is vented and sieve-B is filled. This allows the system to continue to create oxygen in sieve-B while sieve-A is being vented and purged.

At the end of cycle 2 the compressor motor speed is adjusted. The asterisk (*) in Table 2 indicates that the “Adjust” function may happen at the end of cycle 2 only, with the motor speed instead remaining constant during the EQU_Lower state of cycle 1. Motor speed is increased if it takes more than the predetermined length of time (e.g., 6 seconds) to fill a sieve bed 220 to its target pressure. Motor speed is decreased if it takes less than the predetermined length of time (e.g., 6 seconds) to achieve the target pressure in the sieve bed 220. It takes approximately 6 seconds for the zeolite in a sieve bed 220 to adsorb all of the nitrogen from air. To determine whether it takes more or less than 6 seconds (or other predetermined length of time) to achieve target pressure, the elapsed time for both cycles 1 and 2 to complete may be divided by two and then compared to the target time (e.g., 6 seconds). Alternatively, the determination may be made after each cycle, in which case the elapsed time is compared to the target time without first dividing by two.

As shown by way of example in Table 2, the PSA cycle states may further advantageously define the positions of the mix solenoid valve (SV) 250d and the shunt solenoid valve (SV) 250e, as well as those of the O₂ sensor and dump valves 250f, 250g. In this way, the exemplary PSA cycle may fill the product tank 230 according to calculated target volumes of air and oxygen in accordance with the desired setting of the PGS 200. In order to determine the target volumes of air and oxygen of the product tank 230, the control system 400 may include, in addition to the PSA state machine 410, an Update Target Air Mix module 420, a Calculate O₂ Demand module 430, and a Manage Air Mix module 440.

The Update Target Air Mix module 420 may receive a mix setting Set_Mix from a user interface 450 (e.g., a display and input device provided on the housing of the ventilator 300). The mix setting Set_Mix may correspond to a user-selected percentage of oxygen in a mixture of oxygen and ambient air to be delivered by the ventilator 300. For example, a user of the user interface 450 may select a setting such as a numerical setting 0, 1, 2, 3, 4, 5, or 6, which may determine both the mode and the mix setting Set_Mix as shown, for example, in Table 3, below:

TABLE 3

| | | Set_Mix |
|---|---|---|
| Setting | Mode | (oxygen percentage) |
| 0 | Bypass | N/A (21% for ambient air) |
| 1 | Mix | 28% |
| 2 | Mix | 33% |
| 3 | Mix | 40% |
| 4 | Mix | 47% |
| 5 | Mix | 54% |
| 6 | O₂ | N/A (95% sieve bed output) |

The system 10 may then determine, based on a measured airflow demand on the ventilator 300, a maximum percentage of oxygen in the mixture delivered by the ventilator 300 at which the mixture still meets the measured airflow demand. For example, based on the mix setting Set_Mix, the patient breath rate BR in bpm, and the delivered bolus size Bolus in ml, the Update Target Air Mix module 420 may output a value Air_Mix representing the maximum % O₂ that can be produced while still satisfying the airflow demand of the system 10. For example, the Update Target Air Mix module 420 may first calculate the total volume being demanded in one minute, namely, a minute volume MV=BR*Bolus. The Update Target Air Mix module 420 may then set the output value Air_Mix, representing the updated maximum % O₂ that can be produced, as the lesser of Set_Mix and a maximum allowed mix for the calculated MV. That is, the Update Target Air Mix module 420 may set Air_Mix=min (Set_Mix, Max_Allowed_Mix). In this way, the system 10 may override the mix setting Set_Mix with the maximum mix value Max_Allowed_Mix in response to the maximum mix value being less than the user-selected percentage. The maximum allowed mix Max_Allowed_Mix may be found in a lookup table indexed by minute volume MV, such as shown below in Table 4.

TABLE 4

| | Max_Allowed_Mix |
|---|---|
| MV (LPM) | (oxygen percentage) |
| >7.5 | 21% |
| ≤7.5 to >5.3 | 28% |
| ≤5.3 to >3.9 | 33% |
| ≤3.9 to >2.9 | 40% |
| ≤2.9 to >2.3 | 47% |
| ≤2.3 to >1.1 | 54% |
| ≤1.1 | 95% |

For example, if the user setting Set_Mix is 47% oxygen but minute volume MV is 3.5 LPM, upon comparing the Max_Allowed_Mix of 40% to the Set_Mix of 47%, the Update Target Air Mix module 420 will override the Set_Mix value and output 40% as the Air_Mix value (in order to ensure that airflow demands of the system 10 are met. In this way, the patient's ventilation needs may be prioritized over the patient's oxygenation needs for the safety of the patient.

Based on the Air_Mix value output by the Update Target Air Mix module 420, the Calculate O₂ Demand module 430 may calculate a pressure target P_{Target} for the storage tank 230. If the user setting Set_Mix corresponds to user inputs of 0 or 6 (i.e., Bypass or O₂ modes), the Calculate O₂ Demand module 430 may simply set P_{Target}=20 psi. Otherwise, for settings of 1 to 5, the Calculate O₂ Demand module 430 may first calculate an O₂ minute volume demand using the minute volume demand MV=BR*Bolus. For example, the Calculate O₂ Demand module 430 may calculate the O₂ minute volume demand O₂_Demand=MV*(Air_Mix−21%)/74%, which assumes 95% purity of the O₂ produced by the sieve beds 220, such that 95% minus 21% (the percentage of oxygen in room air) equals 74%. The Calculate O₂ Demand module 530 may then calculate P_{Target} according to Table 5, below, which may be derived from a mapping of each range of O² minute volume demand to a corresponding range of pressure targets P_{Target}.

TABLE 5

| O₂ | P_{Target} | P_{Target} |
|---|---|---|
| Demand | Calculation | Range |
| (ml) | (psi) | (psi) |
| ≥1100 | 20 | 20 |
| <1100 to >1000 | ((0.30 * O₂_{_{—}}Demand) − | 17 to 20 |
| | 130 ml)/10 | |
| ≤1000 to >920 | ((0.125 * O₂_{_{—}}Demand) + | 16 to 17 |
| | 45 ml)/10 | |
| ≤920 to >600 | ((0.125 * O₂_{_{—}}Demand) + | 12 to 16 |
| | 45 ml)/10 | |
| ≤600 | 12 | 12 |

Using both the Air_Mix value output by the Update Target Air Mix module 420 and the pressure target P_{Target} output by the Calculate O₂ Demand module 430, the Manage Air Mix module 440 may calculate the total volume of air and O₂ needed to fill the product tank 230. For example, in the case of a 600 ml product tank 230, the Manage Air Mix module 440 may first calculate a total volume of gas needed to fill the product tank 230 Tank_V_{Target}=(((P_{Target}+P_{baro})/(P_{Tank}+P_{baro}))−1)*600 ml+Sieve_O₂_Error, where P_{baro} is barometric pressure as may be determined from a barometric pressure sensor and Sieve_O₂_Error represents an O₂ error derived from the previous target air mix value Air_Mix as described below. In order to calculate a volume of O₂ needed in the product tank 230 O₂_V_{Target} and a volume of air needed in the product tank 230 Air_V_{Target}, the Manage Air Mix module 440 may first calculate O₂_to_Air_Ratio=((Air_Mix)−21%)/(95%−Air_Mix)), where the Air_Mix value may include an appended error correction term Air_Mix+Sieve_Correction representing a correction to adjust the target air mix value Air_Mix when the system is underdelivering or overdelivering oxygen over a period of time. The value may be ±x % (e.g., with x ranging from 0 to 10) and may be incremented or decremented by 1 periodically (e.g., every 100 seconds) when the measured O₂ value is outside 1% of the target air mix value Air_Mix. It is noted that 21% is the percentage of oxygen in room air and 95% is used as an approximate maximum purity of O₂ produced by the sieve beds 220. Using the ratio O₂_to_Air_Ratio and the calculated total volume Tank_V_{Target}, the Manage Air Mix module 440 may then calculate O₂_V_{Target}=Tank_V_{Target}*O₂_to_Air_Ratio/(O₂_to_Air_Ratio+1)−Sieve_O₂_Error and further calculate Air_V_{Target}=Tank_V_{Target}−O₂_V_{Target}. Sieve_O₂_Error may be determined from the volumes of O₂ and air generated during the last fill cycle and the target O₂ to air ratio, e.g., O2_vol−((Air_vol*O2_to_Air_Ratio)/100)+previous Sieve_O2_Error. The purpose of this value is to verify that the O2 and air volumes were generated in correct proportion to each other in the previous cycle, and if not, to adjust the next air and O2 target volumes Air_V_{Target} and O2_V_{Target} accordingly.

Referring again to the PSA state machine 410 described in relation to Tables 1 and 2, the “Mix” settings of the Mix SV may be determined as follows by way of example. As noted above, the system will stay in the FILL state until the pressure in both the storage tank 230 and sieve A reaches the target pressure (i.e., P_{Tank}>P_{Target} & P_{Comp}>P_{Target}), or 10 seconds elapses (i.e., “Time Out”), whichever occurs first. Thus, the sieve bed pressure P_{Comp} may first be allowed to rise to the level of the pressure in the storage tank 230 P_{Tank}, during which the Manage Air Mix module 440 may calculate Air_V_{Target} and O₂_V_{Target} as described above. The air and O₂ target volumes Air_V_{Target} and O₂_V_{Target} may be recalculated every time the FILL state is executed. Then, using these target volumes, the system may decide when to switch the MixSV. If the measured volume of oxygen Measured_O₂_V is greater than O₂_V_{Target}+30 ml, the mix solenoid valve 250d (Mix_SV) may be set to Air to begin delivering air from the compressor 210 into the product tank 230. (The FILL state may be initialized to start with the Mix SV 250d switched to air.) The Manage Air Mix module 440 may again calculate Air_V_{Target} and O₂_V_{Target} as described above. If the measured volume of air Measured_Air_V is greater than Air_V_{Target}, then the Mix SV 250d may switch to O₂ delivery to begin filling the product tank 230 from the sieve beds 220. The target pressure P_{Target} may then be established by the Calculate O₂ Demand module 430. In this way, the FILL state may build pressure until both the product tank and compressor pressure both exceed the target pressure, while also delivering the correct mixture of air and O₂ to the product tank 230. It is noted that the measured volumes of oxygen and air may be determined by integrating the flow to the storage tank 230 while the mix valve 250d is set to either oxygen or air, as represented by the Sigma v′ dt box in FIG. 4.

The various functionality, operations, and control systems described herein, as may be performed by a controller of the PGS 200 and/or a controller of the ventilator 300 for example, may be implemented with a programmable integrated circuit device such as a microcontroller or control processor. Broadly, the device may receive certain inputs, and based upon those inputs, may generate certain outputs. The specific operations that are performed on the inputs may be programmed as instructions that are executed by the control processor. In this regard, the device may include an arithmetic/logic unit (ALU), various registers, and input/output ports. External memory such as EEPROM (electrically erasable/programmable read only memory) may be connected to the device for permanent storage and retrieval of program instructions, and there may also be an internal random access memory (RAM). Computer programs for implementing any of the disclosed functionality of the controller(s) may reside on such non-transitory program storage media, as well as on removable non-transitory program storage media such as a semiconductor memory (e.g., IC card), for example, in the case of providing an update to an existing device. Examples of program instructions stored on a program storage medium or computer-readable medium may include, in addition to code executable by a processor, state information for execution by programmable circuitry such as a field-programmable gate arrays (FPGA) or programmable logic device (PLD).

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A computer program product comprising one or more non-transitory program storage media on which are stored instructions executable by a processor or programmable circuit to perform operations for controlling a portable gas source connected to a ventilator, the operations comprising:
receiving, via a user interface, a mix setting corresponding to a user-selected percentage of oxygen in a mixture of oxygen and ambient air to be delivered by the ventilator;
determining, based on a measured airflow demand on the ventilator, a maximum percentage of oxygen in the mixture at which the mixture still meets the measured airflow demand; and
overriding the mix setting with the maximum mix value in response to the maximum mix value being less than the user-selected percentage.

2. The computer program product of claim 1, wherein said determining includes calculating a total volume being demanded in one period based on a breath rate and a delivered bolus size and determining the maximum percentage of oxygen based on the calculated total volume.

3. The computer program product of claim 1, wherein the operations further comprise calculating, based on the maximum mix value, a pressure target for a storage tank of the mixture of oxygen and ambient air to be delivered by the ventilator.

4. The computer program product of claim 3, wherein the operations further comprise calculating, based on the maximum mix value and the pressure target, a total volume of gas needed to fill the product tank.

5. The computer program product of claim 4, wherein the operations further comprise calculating, based on the maximum mix value and the total volume of gas needed to fill the product tank, a total volume of air needed to fill the product tank and a total volume of oxygen needed to fill the product tank.

6. The computer program product of claim 5, wherein the operations further comprise controlling a solenoid valve based on the total volume of air needed to fill the product tank and the total volume of oxygen needed to fill the product tank.

7. A method of controlling a portable gas source connected to a ventilator, the method comprising:
receiving, via a user interface, a mix setting corresponding to a user-selected percentage of oxygen in a mixture of oxygen and ambient air to be delivered by the ventilator;
determining, based on a measured airflow demand on the ventilator, a maximum percentage of oxygen in the mixture at which the mixture still meets the measured airflow demand; and
overriding the mix setting with the maximum mix value in response to the maximum mix value being less than the user-selected percentage.

8. The method of claim 7, wherein said determining includes calculating a total volume being demanded in one period based on a breath rate and a delivered bolus size and determining the maximum percentage of oxygen based on the calculated total volume.

9. The method of claim 7, further comprising calculating, based on the maximum mix value, a pressure target for a storage tank of the mixture of oxygen and ambient air to be delivered by the ventilator.

10. The method of claim 9, further comprising calculating, based on the maximum mix value and the pressure target, a total volume of gas needed to fill the product tank.

11. The method of claim 10, further comprising calculating, based on the maximum mix value and the total volume of gas needed to fill the product tank, a total volume of air needed to fill the product tank and a total volume of oxygen needed to fill the product tank.

12. The method of claim 11, further comprising controlling a solenoid valve based on the total volume of air needed to fill the product tank and the total volume of oxygen needed to fill the product tank.

13. A system for controlling a portable gas source connected to a ventilator, the system comprising:
a user interface by which a user may input a mix setting corresponding to a user-selected percentage of oxygen in a mixture of oxygen and ambient air to be delivered by the ventilator; and
one or more processors operable to determine, based on a measured airflow demand on the ventilator, a maximum percentage of oxygen in the mixture at which the mixture still meets the measured airflow demand and to override the mix setting with the maximum mix value in response to the maximum mix value being less than the user-selected percentage.

14. The system of claim 13, wherein said determining includes calculating a total volume being demanded in one period based on a breath rate and a delivered bolus size and determining the maximum percentage of oxygen based on the calculated total volume.

15. The system of claim 13, wherein the one or more processors are further operable to calculate, based on the maximum mix value, a pressure target for a storage tank of the mixture of oxygen and ambient air to be delivered by the ventilator.

16. The system of claim 15, wherein the one or more processors are further operable to calculate, based on the maximum mix value and the pressure target, a total volume of gas needed to fill the product tank.

17. The system of claim 16, wherein the one or more processors are further operable to calculate, based on the maximum mix value and the total volume of gas needed to fill the product tank, a total volume of air needed to fill the product tank and a total volume of oxygen needed to fill the product tank.

18. The system of claim 17, wherein the one or more processors are further operable to control a solenoid valve based on the total volume of air needed to fill the product tank and the total volume of oxygen needed to fill the product tank.

19. The system of claim 13, wherein the user interface is provided on a housing of the ventilator.

20. The system of claim 13, wherein the one or more processors are provided in the portable gas source.
